(19) United States
(12) Patent Application Publication
(10) Pub. No.: US 2004/0265381 A1
(43) Pub. Date: Dec. 30, 2004

(54) Anti-asthmatic drug (asmakure) from indigenous herbs to cure the disease asthma

(76) Inventors: Dilip S. Shanghvi, Mumbai (IN); Ashish P. Mungre, Mumbai (IN); Yashoraj R. Zala, Mumbai (IN)

Correspondence Address: TIMOTHY J MARTIN, PC, 9250 W 5TH AVENUE, SUITE 200, LAKEWOOD, CO, 80226, US

(21) Appl. No.: 10/492,070

(22) PCT Filed: Oct. 8, 2002
(86) PCT No.: PCT/IN02/00203

(30) Foreign Application Priority Data
Oct. 8, 2001 (IN) 984/MUM/2001
Apr. 9, 2002 (WO) PCT/IN02/00107

(51) Int. Cl. A61K 9/22

(52) U.S. Cl. 424/468

(57) ABSTRACT

Asthma is defined as a chronic inflammatory disorder of the airways of the respiratory organ. It is characterized by reversible airflow obstruction causing cough, wheeze, chest lightness and shortness of breath. The inflammation of bronchial wall together with increased eosinophilis and other inflammatory products of the mast cells and lymphocytes further induce the hyper responsiveness of the bronchi so that it in turn, narrows more rapidly in response to a wide range of stimuli. Asmakure the anti-asthma drug has properties with proven pharmacological use for alleviating comman cold and persistent cough and finally building up of immunity against recurrence of asthma. One of the ingredients Adhatoda Vasica Nees (Basak) has a definite expectorant action. In acute bronchitis, it is found to afford immediate relief especially when the sputum is thick and tenacious. The depression of the Vagal terminals further relieves irritation and spasm of the bronchioles.

The present invention relates to an antispasmodic agent spaced drug delivery system that provides spaced drug delivery of oxybutynin wherein oxybutynin is released in a pulse initially and then at one or more predetermined time intervals.

### BACKGROUND OF THE INVENTION

Oxybutynin chloride, [α-cyclohexyl-α-hydroxybenzeneacetic acid 4-(diethylamino)-2-butynyl ester hydrochloride] disclosed in the British Patent No. 940,540 and U.S. Pat. No. 4,870,074 is a musculotropic antispasmodic drug with moderate anticholinergic, systemic analgesic and local anaesthetic action. Its relaxant effect on smooth muscle is based on antagonism of a process distal to the neuromuscular junction (papaverine-like effect) and on anticholinergic action on the blockage of muscarine-type receptors. Oxybutynin chloride has been in clinical use for twenty years and it is indicated for the relief of symptoms associated with voiding in patients with an uninhibited neurogenic and reflex neurogenic bladder.

Oxybutynin is rapidly absorbed from the gastrointestinal tract following oral administration and its pharmacological action starts within one hour. The duration of action of the drug is three to six hours. It has a half-life of less than 2 hours. The usual dose in the management of incontinence is repeated doses of 5 mg tablets from two-to-four times a day. This is difficult to achieve as it requires rigid patient compliance and also it is cost ineffective.

The concept of using a spaced drug delivery system for oxybutynin, wherein oxybutynin is released immediately initially and then as a pulse at one or more predetermined time intervals to eliminate the need for multiple dosing in the therapy of urinary incontinence is neither known in the art nor suggested by any prior art reference.

Further the concept of providing a spaced drug delivery system for oxybutynin wherein predetermined amount of oxybutynin is released as a pulse initially and then predetermined number of pulses of predetermined amounts of oxybutynin release provided for optimum therapy of urinary incontinence is neither known in the art nor suggested by any prior art reference.

U.S. Pat. No. 3,247,066 ('066) claims a controlled release dosage form comprising a solid bead covered by a rupturable plastic, non-toxic, insoluble, non-digestible film coating which is inert to the gastrointestinal fluid and permeable to diffusion of water, the bead containing a uniform dispersion of the medicament in a water-swellable colloid, the thickness of the coating and swellability of the bead being such that on prolonged exposure to gastrointestinal fluid, diffusion of water takes place through the coating into the bead, causing the bead to swell up and build up pressure exceeding the cohesive strength of the coating thereby resulting in outward rupture of the coating and release of the medicament from the bead into the gastrointestinal fluid. However, in the systems disclosed and exemplified in the '066 patent the swellable colloid used is gelatin that exhibits only a small to moderate degree and rate of swelling and fails to provide the formulator a desired degree of swelling to cause the release to occur as a pulse at about a predetermined time period. Also the patent does not suggest the use of antispasmodics as a therapeutic class, in the subject invention.

U.S. Pat. No. 5,654,009 ('009) discloses an intramuscularly or subcutaneously administered delayed action preparation comprising a core portion containing the drug and a hydratable swelling polymer, and an outer membrane of a biodegradable high molecular weight substance surrounding the core. The water penetrating through the outer layer into the core causes swelling of the hydratable swelling polymer present in the core, thereby causing an explosion of the outer membrane. This system claims an improvement over prior art in that it is capable of causing the outer membrane to explode at a predetermined time. However, the system of the '009 patent is neither particularly meant to nor is it capable of releasing the drug thereafter at a rapid rate when desired.

Also the patent does not suggest the use of antispasmodics as a therapeutic class, in the subject invention.

U.S. Pat. No. 5840754 discloses a method of lessening the incidence of side-effects in a patient comprising administering oxybutynin tablet dosage form that can deliver oxybutynin over 24 hours to the patient at a substantially controlled and sustained rate to provide control on desired plasma oxybutynin concentration and reduce peak plasma concentration. The patent teaches a person skilled in the art that pulse release of oxybutynin would be undesirable.

We have found a spaced drug delivery system for oxybutynin or its pharmaceutically acceptable salts comprising (a) an immediate release composition releasing oxybutynin or its pharmaceutically acceptable salts immediately initially and (b) timed pulse release composition(s) releasing oxybutynin or its pharmaceutically acceptable salts in a pulse at one or more predetermined time intervals. Surprisingly the spaced drug delivery system is suitable for twice-a-day or once-a-day therapy of urinary incontinence.

No prior art reference suggests the use of such a spaced drug delivery system with programmed pulse release of oxybutynin for twice-a-day or once-a-day therapy of urinary incontinence.

### OBJECT OF THE INVENTION

It is an object of the present invention to provide an antispasmodic agent spaced drug delivery system that provides spaced drug delivery of oxybutynin for twice-a-day or once-a-day therapy i.e the spaced drug delivery system is such that oxybutynin is released initially and then at one or more predetermined time intervals in a pulse.

More specifically it is a specific object of the present invention to provide an antispasmodic agent spaced drug delivery system capable of programmed delivery or release in a predetermined number of pulses of predetermined amounts of oxybutynin at spaced predetermined time intervals for optimum therapy of urinary incontinence.

### BRIEF DESCRIPTION OF THE DRAWINGS

FIG. 1 shows the plasma concentration vs time profile obtained upon administration of an embodiment of the spaced drug delivery system of oxybutynin chloride of the present invention in comparison to commercially available Ditropan XL tablets.

### DESCRIPTION OF THE INVENTION

The present invention discloses a novel concept of spaced drug delivery system for oxybutynin. The spaced drug delivery system of the present invention can be administered in a single dose to the patient to provide therapeutically effective plasma levels of oxybutynin over a period of 12 hours to 24 hours by delivering at spaced time intervals the oxybutynin in pulses. The need for the patient to take multiple dosages during this interval is obviated.

The present invention specifically provides an antispasmodic agent spaced drug delivery system comprising an immediate release composition releasing oxybutynin immediately as a pulse and one or more timed pulse release composition(s) releasing oxybutynin in a pulse at predetermined time intervals.

More specifically, the present invention provides an antispasmodic agent spaced drug delivery system comprising;

(a) an immediate release composition comprising oxybutynin or its pharmaceutically acceptable salts and pharmaceutically acceptable excipients;

(b) a timed pulse release composition releasing oxybutynin or its pharmaceutically acceptable salts in a pulse at about a predetermined time; and

(c) optionally further comprising timed pulse release composition(s) as defined in (b) above, and releasing oxybutynin or its pharmaceutically acceptable salts in a pulse at about a predetermined time which is different from that of the timed pulse release composition defined in (b).

The spaced drug delivery system of the present invention comprises therapeutically effective amount of oxybutynin for twice-a-day or once-a-day therapy. Preferably oxybutynin chloride may be present in amounts in the range from about 2.5 mg to 30 mg, more preferably from about 5 mg to 15 mg.

The oxybutynin amount may be the same in the immediate release and the timed pulse release compositions. For example, in a particular embodiment of a spaced drug delivery system comprising 15 mg oxybutynin chloride; 5 mg may be present in the immediate release composition, 5 mg may be present in a first timed pulse release composition releasing the 5 mg at a first predetermined time and 5 mg may be present in a second timed pulse release composition releasing the 5 mg at a second predetermined time.

The oxybutynin amount may be different in the immediate release and the timed pulse release compositions. For example, in a particular embodiment of a spaced drug delivery system comprising 10 mg oxybutynin chloride; 2.5 mg may be present in the immediate release composition, 4 mg may be present in a first timed pulse release composition releasing the 4 mg at a first predetermined time and 3.5 mg may be present in a second timed pulse release composition releasing the 3.5 mg at a second predetermined time.

The spaced drug delivery system can be designed to release predetermined amounts of oxybutynin as a pulse initially and then at predetermined time intervals to provide optimum therapy. The number of pulses of oxybutynin release can also be selected to optimize the therapy. The spaced drug delivery system of the present invention thus provides ease and flexibility in providing desired plasma levels of oxybutynin for optimum therapy.

Immediate release compositions are well known in the art and the immediate release composition of the spaced drug delivery system of the present invention may be provided according to the known art. Similarly the timed pulse release composition may be provided using known art but preferably the composition is provided in accordance with preferred embodiments of the present invention as described herein below.

The preferred antispasmodic agent spaced drug delivery system of the present invention comprises;

(a) an immediate release composition comprising oxybutynin or its pharmaceutically acceptable salts and pharmaceutically acceptable excipients;

(b) a timed pulse release composition comprising a core comprising oxybutynin or its pharmaceutically acceptable salts, a swelling agent that swells to at least twice its volume upon imbibing water from the environment, and optionally, water-soluble compound(s) for inducing osmosis; and a coat surrounding the core wherein the coat comprises coating agents selected and used in amounts such that the coat ruptures or bursts to release in a pulse, the oxybutynin or its pharmaceutically acceptable salts at about a predetermined time; and

(c) optionally further comprising timed pulse release composition(s) as defined in (b) above, but which release(s) oxybutynin or its pharmaceutically acceptable salts in a pulse at about a predetermined time which is different than that of the timed pulse release composition defined in (b).

### PREFERRED AND MOST PREFERRED EMBODIMENTS

The preferred timed pulse release composition of the present invention comprising a core comprising oxybutynin or its pharmaceutically acceptable salts, a swelling agent, optionally, water-soluble compounds for inducing osmosis and a coat surrounding the core, is provided, wherein upon imbibing fluid from the surrounding the core swells, and the coat ruptures or bursts to release in a pulse, the oxybutynin or its pharmaceutically acceptable salts at about a predetermined time. The coat of the preferred timed pulse release composition of the present invention ruptures in a reliable manner. In 36 tablets out of a total of 36 tablets the coat ruptured or burst to release oxybutynin or its pharmaceutically acceptable salts in a pulse at about a predetermined time when tested by subjecting the tablets to USP dissolution test using an aqueous media at 37±0.5° C., in a USP Type I or Type II apparatus at an rpm selected from the range of about 50 rpm to about 100 rpm. Further the 36 out of the 36 tablets rupture within about ±50% of the predetermined time.

The term “release in a pulse” refers to release characteristic of conventional tablets and capsules that are devoid of design characteristics that result in slow, extended, controlled or retarded release of the therapeutically active agent when tested by standard in-vitro testing methods. For example, in a particular embodiment where the predetermined time of pulse release is about 4 hours, the “release of oxybutynin or its pharmaceutically acceptable salts in a pulse” comprises release, of not more than 10% of the active ingredient at 3 hours and 45 min and at least 70% of the active ingredient at 2 hrs after the coat ruptures or bursts, which is about 6 hours after the start of the dissolution test, when tested by subjecting the tablets to USP dissolution test using pH 4.5 acetate buffer at 37±0.5° C., in a USP Type II apparatus at an rpm of 50.

The swelling agent that may be used in the preferred composition of the present invention is selected from hydrophilic polymers. The hydrophilic polymers may be of plant, animal, mineral or synthetic origin. Suitable swellable polymers for use in the present invention include (A) cellulose derivatives such as C₁₋₄ alkyl celluloses like methyl cellulose and ethyl cellulose; hydroxy C₁₋₄ alkyl celluloses such as hydroxymethyl cellulose, hydroxyethyl cellulose, hydroxypropyl cellulose, and the like; hydroxy C₁₋₄ alkyl C₁₋₄ alkyl celluloses such as hydroxypropyl methylcellulose, hydroxypropyl ethylcellulose and the like; carboxy C₁₋₄ alkyl celluloses such as carboxymethyl cellulose, carboxyethyl cellulose, and their alkali salts; and the like, (B) vinylpyrrolidone polymers such as polyvinyl pyrrolidone, crosslinked polyvinyl pyrrolidone or crospovidone and the like, (C) copolymers of vinyl pyrrolidone and vinyl acetate, (D) gums of plant, animal, mineral or synthetic origin such as (i) agar, alginates, carrageenan, furcellaran obtained from marine sources, (ii) guar gum, gum Arabic, gum tragacanth, karaya gum, locust bean gum obtained from terrestrial plants, (iii) microbial polysaccharides such as dextran, gellan gum, rhamsan gum, welan gum, xanthan gun, and (iv) synthetic or semi-synthetic gums such as propylene glycol alginate, hydroxypropyl guar and modified starches like sodium starch glycolate. The swelling agent used in the present invention is preferably a combination of the agents mentioned above. Often, a combination of two agents provides a controlled swelling thereby causing the coat or core to rupture or burst open at a predetermined time after oral administration of the delivery system.

The swelling agent that may be used in the present invention may comprise one or more swellable hydrophilic polymers. The quantity or relative proportion of the polymers is subject to considerable variation. However, a sufficient quantity of the material is present in the core to provide, upon uptake of water, a swelling pressure in excess of the cohesive strength of the coating surrounding the tablet or core. Preferably, the polymers are employed in the dry state or in a form that has substantial capacity for water uptake. Examples of swellable hydrophilic polymers that may be used in the timed pulse release composition of the present invention as the swelling agent include vinylpyrrolidone polymers such as povidone, or crosslinked polyvinylpyrrolidone such as crospovidone; cellulose and cellulose derivatives such as microcrystalline cellulose, methylcellulose, ethylcellulose, hydroxypropylcellulose, hydroxypropyl methylcellulose, carboxyalkyl celluloses or crosslinked carboxyalkylcelluloses and their alkali salts; sodium starch glycolate, starch and starch derivatives, ion-exchange resins and mixtures thereof. Preferably, the swelling agent used comprises a swelling agent that swells considerably but does not form a strong gel, and may be selected from the group comprising crosslinked sodium carboxymethyl cellulose, crosslinked polyvinylpyrrolidone and sodium starch glycolate.

The alkali salt of crosslinked carboxyalkyl cellulose, i.e. crosslinked sodium carboxymethyl cellulose, also known as croscarmellose sodium or Ac-Di-Sol, is available commercially as Nymcel® ZSX, Pharmacel® XL, Primellose® or Solutab®. The amount of swelling agent that may be used is dependent on the desired time of rupture of the timed pulse release coat, nature and amounts of other components in the core, as well as the composition and thickness of the coat. Generally, croscarmellose sodium may be used as the polymeric swelling agent in an amount ranging from about 1% to about 95% by weight of the core, preferably from about 2% to about 40% by weight of the core, more preferably from about 5% to about 20% by weight of the core.

Vinyl pyrrolidone polymers or polyvinyl pyrrolidone (PVP), also referred to as Povidone, are synthetic polymers consisting essentially of linear 1-vinyl-2-pyrrolidinone groups, the degree of polymerization of which results in polymers of various molecular weights, the molecular weight ranging between 2500 and 3,000,000 Daltons. PVP is commercially available as Kollidon® (BASF), Plasdone® and Peristone® (General Aniline). PVP is classified into different grades on the basis of its viscosity in aqueous solution. Different grades of PVP available are PVP K-12, PVP K-15, PVP K-17, PVP K-25, PVP K-30, PVP K-60, PVP K-90 and PVP K-120. The K-value referred to in the above nomenclature is calculated from the viscosity of the PVP in aqueous solution, relative to that of water. Crospovidone or cross-PVP, the synthetic crosslinked homopolymer of N-vinyl-2-pyrrolidinone, may also be used as a swellable hydrophilic polymer. It is commercially available as Kollidon CL and Polyplasdone XL, and has a molecular weight higher than 1,000,000 Daltons. Crospovidone may be used as the swellable hydrophilic polymers in an amount ranging from about 2% to about 5% by weight of the core. The preferred vinyl pyrrolidone polymer for use as a swellable hydrophilic polymer is PVP K-30, having an approximate molecular weight of 50,000 Daltons. It may be used in an amount ranging from about 0.5% to about 5% by weight of the core, more preferably from about 1% to about 2% by weight of the core.

Sodium starch glycolate, the sodium salt of carboxymethyl ether of starch, may also be used as the polymeric swelling agent. It has a molecular weight ranging between 500,000 and 1,000,000 Daltons, and is commercially available as Explotab and Primojel. Sodium starch glycolate may be used in the present invention in an amount ranging from about 0.5% to about 40% by weight of the core, preferably from about 2% to about 40% by weight of the core, more preferably from about 2% to about 10% by weight of the core.

Preferably, the core in the timed pulse release composition contains a wicking agent. The term wicking agent as used herein implies a broader definition than a conventional wicking agent and includes any pharmaceutical excipient that provides influx of water into the core by any suitable mechanism, preferably by capillary action as is typical of conventional wicking agents. Materials suitable for use as wicking agents in the timed pulse release composition include, but are not limited to, colloidal silicon dioxide, kaolin, titanium dioxide, fumed silicon dioxide, alumina, sodium lauryl sulfate, microcrystalline cellulose, low molecular weight polyvinyl pyrrolidone, bentonite, magnesium aluminum silicate, and the like. The timed pulse release composition of the antispasmodic agent spaced drug delivery system of the present invention may be optimized to obtain the reliable manner of rupture without the use of a wicking agent. However, the use of a wicking agent has been found to be useful to make the task of optimization to obtain the reliable manner of rupture easier.

Microcrystalline cellulose (MCC) used in the preferred embodiment as the wicking agent, is made up of a chain of about 250 glucose molecules in the form of a microcrystal, consisting primarily of crystallite aggregates obtained by removing amorphous regions of a pure cellulose source material by hydrolytic degradation using mineral acid. MCC has an average molecular weight of about 36,000 Daltons and is available in various grades, which differ in bulk density, particle size and moisture content. It is commercially available as Vivapur®, Avicel®, Vivacel®, Emcocel®, Fibrocel® and Tabulose®. Avicel® PH 101, having a mean particle size of 50 μm, i.e. 1% or less of the particles are retained on a # 60 sieve (as defined by ASTM, American Society for Testing and Materials), and 30% or less of the particles are retained on a # 200 sieve (as defined by ASTM), and having a moisture content 5% and Avicel® PH 102, having a mean particle size of 100 μm, i.e. 8% or less of the particles are retained on a # 60 sieve (as defined by ASTM, American Society for Testing and Materials), and 45% or more of the particles are retained on a # 200 sieve (as defined by ASTM), and having a moisture content of 5%, are used in more preferred embodiments of the timed pulse release composition.

Preferably, the wicking agent comprises a mixture of microcrystalline cellulose (MCC) and colloidal silicon dioxide. In preferred embodiments of the present invention made by granulation techniques, MCC is added intragranularly and extragranularly wherein it is present in an amount ranging from about 30% to about 90% by weight of the core, more preferably about 40% to about 60% by weight of the core intragranularly and is present along with colloidal silicon dioxide extragranularly preferably in an total amount ranging from about 1% to about 10% by weight of the core, more preferably about 2% to about 5% by weight of the core.

Water-soluble compounds suitable for inducing osmosis, i.e. osmotic agents or osmogents are generally used in the core of the timed pulse release composition when the drug itself does not exert sufficient osmotic pressure in order to imbibe fluid from the surroundings. Osmogents that may be present in the core of the timed pulse release composition include all pharmaceutically acceptable and pharmacologically inert water-soluble compounds referred to in the pharmacopocias such as United States Pharmacopoeia, as well as in Remington: The Science and Practice of Pharmacy, edition 20, Lippincott Williams and Wilkins, Philadelphia (2000). Pharmaceutically acceptable water-soluble salts of inorganic or organic acids, or non-ionic organic compounds with high water solubility, e.g. carbohydrates such as sugar, or amino acids, are generally preferred. The examples of agents used for inducing osmosis include inorganic salts such as magnesium chloride or magnesium sulfate, lithium, sodium or potassium chloride, lithium, sodium or potassium hydrogen phosphate, lithium, sodium or potassium dihydrogen phosphate, salts of organic acids such as sodium or potassium acetate, magnesium succinate, sodium benzoate, sodium citrate or sodium ascorbate; carbohydrates such as mannitol, sorbitol, arabinose, ribose, xylose, glucose, fructose, mannose, galactose, sucrose, maltose, lactose, raffinose; water-soluble amino acids such as glycine, leucine, alanine, or methionine; urea and the like, and mixtures thereof. The amount of osmogents that may be used depends on the particular osmogent that is used and may range from about 1% to about 60% by weight of the core.

In addition to the above ingredients, the core of the timed pulse release composition may optionally contain pharmaceutically acceptable excipients such as binders, disintegrants, lubricants and the like. Examples of binders used commonly include starch, gelatin, sugars like sucrose, glucose, dextrose, molasses and lactose; acacia, sodium alginate, cellulose derivatives like methyl cellulose, ethyl cellulose, carboxymethyl cellulose and the like; polymers such as polyvinyl pyrrolidone, Veegum, polyethylene glycol, waxes and the like. Examples of lubricants that may be used in the timed pulse release composition include talc, magnesium stearate, calcium stearate, aluminium stearate, stearic acid, hydrogenated vegetable oils, colloidal silicon dioxide, polyethylene glycol, cellulose derivatives such as carboxyalkyl cellulose and its alkali salts, or mixtures thereof. Hydrophobic or water insoluble lubricants may reduce the water imbibing properties of the core whereas hydrophilic or water soluble lubricants do not, and are preferred. A more preferred lubricant is colloidal silicon dioxide. A mixture of colloidal silicon dioxide and magnesium stearate may be used as the preferred lubricant. More preferred embodiments use a combination of microcrystalline cellulose and colloidal silicon dioxide as the wicking agents, with colloidal silicon dioxide also functioning as a lubricant. Colloidal silicon dioxide is available commercially as Aerosil® from Degussa-Huls, Nippon and Fischer GmbH. The preferred colloidal silicon dioxide lubricant is Aerosil®, with an approximate external surface area of 200 m²/g. The colloidal silica may be used in amounts in the range of about 0.5% to about 5% by weight of the core.

The coat surrounding the core is substantially impermeable to the drug and does not release substantial amount of the drug, until it ruptures or bursts at a predetermined time after oral administration of the delivery system. The coating agents that may be used in the present invention are selected from among water insoluble polymers, hydrophobic compounds, hydrophilic non-polymeric compounds and hydrophilic polymers that may be of plant, animal, mineral or synthetic origin. Suitable coating agents include among others cellulose derivatives such as cellulose acetate phthalate, hydroxypropyl methylcellulose, hydroxypropyl cellulose, hydroxypropyl ethylcellulose, ethyl cellulose, methyl cellulose, microcrystalline cellulose, carrageenan, or mixtures thereof, and the like; methacrylic acid and methacrylate esters such as anionic and cationic polymers of methacrylic acid, copolymers of methacrylates, copolymers of acrylates and methacrylates, copolymers of ethacrylate and methylmethacrylate, polyvinylacetate phthalate, waxes such as beeswax, carnauba wax, and the like, glyceryl monostearate, stearic acid, palmitic acid, glyceryl monopalmitate, cetyl alcohol and the like, or mixtures thereof. The term coating agent as used herein also includes plasticizers that are used in coating compositions and are known to those skilled in the art. The plasticizer may be a low molecular weight compound or may itself be a polymer. Preferably the coating agent comprises a mixture of at least two coating agents. The time of release of oxybutynin or its pharmaceutically acceptable salts from the timed release composition may be varied by varying the ratio of the two or more coating agents. Preferably the coating agent is a mixture of a water insoluble polymer and a water soluble compound selected from a water soluble plasticizer and water soluble polymer. In a preferred embodiment of the present invention, a mixture of ethyl cellulose and hydroxypropyl methylcellulose (HPMC) is used as the coating agent. Preferably the ethyl cellulose and hydroxypropyl methylcellulose is used in mixture in a ratio suitable to cause opening of the coat at a predetermined time after oral administration of the delivery system. The two polymers ethyl cellulose: hydroxypropyl methylcellulose may be preferably used in ratios from about 0:20 to about 20:0 of ethyl cellulose : hydroxypropyl methylcellulose, more preferably the two polymers may be used in ratios from about 4:1 to about 2:1 of ethyl cellulose: hydroxypropyl methylcellulose.

A more preferred embodiment of the present invention is in the form of a spaced drug delivery system comprising an immediate release composition that releases oxybutynin or its pharmaceutically acceptable salts within 30 minutes after oral administration, a first timed pulse release composition that releases oxybutynin or its pharmaceutically acceptable salt in a pulse at about a predetermined time after oral administration wherein the time of release lies in the range of 3 to 6 hours, and a second timed pulse release composition that releases oxybutynin or its pharmaceutically acceptable salt in a pulse at about a predetermined time after oral administration wherein the time of release lies in the range of 6 to 10 hours. In a particular preferred embodiment the amounts of oxybutynin released at 0 hours, 3 to 6 hours and 6 to 10 hours may at each of these times of release represent 33.33% by weight of the total amount of oxybutynin in the spaced drug delivery system. In another preferred embodiment the amounts of oxybutynin released at 0 hours, 3 to 6 hours and 6 to 10 hours may at each of these times of release represent 25%, 40% and 35% by weight of the total amount of oxybutynin in the spaced drug delivery system.

In a particularly preferred embodiment of the present invention the core of the timed pulse release composition comprises of the following:

TABLE 1

| | | Range of amount used |
|---|---|---|
| | | as percent by weight of |
| Components | Name | core |
| Antispasmodic | Oxybutynin chloride | 2-5% |
| agent | | |
| Swelling agent | Croscarmellose sodium | 5-15% |
| Wicking agent | Microcrystalline cellulose | 40-60% |
| | Colloidal silicon dioxide | 1-30% |
| Water soluble | Lactose | 15-25% |
| compound | | |

and optionally other pharmaceutical excipients.

The core of the above timed pulse release composition as in Table 1 may be surrounded by a coat comprising of coating agent comprising a mixture of ethyl cellulose hydroxypropyl methylcellulose in the ratios by weight of 4:1 to 2:1, most preferably 3:1. The coating agent may be added in an amount from about 12 to 15% by weight of the core to release the oxybutynin or its pharmaceutically acceptable salts in a pulse at about 4 hours. Alternatively the coating agent may be added in an amount from about 18 to 22% by weight of the core to release oxybutynin or its pharmaceutically acceptable salts in a pulse at about 8 hours. In a highly preferred embodiment the spaced drug delivery system for oxybutynin or its pharmaceutically acceptable salts comprise the immediate release composition, a first timed pulse release composition providing pulse release at 4 hours and a second timed pulse release composition providing pulse release at 8 hours.

The antispasmodic agent spaced drug delivery system of the present invention may be prepared by methods well known to those skilled in the art. The timed pulse release composition may be prepared in the form of coated tablets or coated pellets or coated granules by conventional means known in the art. The immediate release composition may be prepared by conventional means known in the art as a separate physical component of the system, for example separate or physically distinct particles, granules, beads or tablets. Alternatively, the immediate release composition may be in the form of a layer associated with the timed pulse release composition either as a laminar layer or as a layer that surrounds the timed pulse release coat. When the composition is present as a layer or coat over the timed pulse release core, it is formed by conventional techniques known to a person skilled in the art such as powder layering and compression coating. The active ingredient(s) may alternatively be dissolved in a suitable solvent and sprayed on the coated core to form the immediate release layer.

The following examples do not limit the scope of the invention and is presented as illustrations.

### EXAMPLE 1

The antispasmodic agent spaced drug delivery system of the present invention was prepared as per the formula in Table 2 below.

TABLE 2

| | Quantity (mg) | Percent (%) w/w. |
|---|---|---|
| | (cores for immediate release | |
| | tablet, tablet with 4 | |
| | hour and 8 hour timed pulse | |
| Ingredients | release composition) | |
| Intragranular | | |
| Oxybutynin chloride | 3.3 | 3.66 |
| Microcrystalline cellulose | 50.0 | 55.56 |
| (Avicel PH 101) | | |
| Lactose monohydrate | 18.2 | 20.22 |
| Crocarmellose sodium (Ac-Di-Sol) | 9.0 | 10.0 |
| Maize starch (as 10% starch paste) | 5.0 | 5.56 |
| Extragranular | | |
| Microcrystalline cellulose | 2.0 | 2.22 |
| (Avicel PH 102) | | |
| Colloidal silicon dioxide | 2.0 | 2.22 |
| (Aerosil 200) | | |
| Magnesium stearate | 0.5 | 0.56 |
| Total | 90 | 100.0 |

The cores for the immediate release tablets, tablets releasing oxybutynin at a predetermined time of 4 hours and the tablets releasing oxybutynin at a predetermined time of 8 hrs were prepared by the method of preparation as described herewith. Oxybutinin chloride, Avicel PH 101, lactose monohydrate and croscarmellose sodium were sifted through a suitable sieve and mixed in a rapid mixer granulator. The dry powder blend was then granulated using 10% starch paste, followed by wet milling the mass through a fitz mill. The granules so obtained were dried to moisture content of 3-4%. The dry granules were then milled in a fitz mill through a 1.55 mm screen, followed by sifting of the granules through a #16 sieve (as defined by American Society for Testing and Materials, ASTM). These granules of oxybutynin chloride were then mixed with Avicel PH 102, colloidal silicon dioxide and magnesium stearate and the lubricated mixture thus obtained was compressed on a rotary compression machine using round shaped punches.

The immediate release tablet cores were not coated while the tablet cores for releasing the drug at a predetermined time intervals were coated using the coating composition given in Table 3 below.

TABLE 3

| Ingredients | Quantity (% w/w) |
|---|---|
| Ethyl cellulose | 3.75 |
| Hydroxypropyl methylcellulose (HPMC 50) | 1.25 |
| Dichloromethane | q.s. |
| Methanol | q.s. |

The core tablets were coated in a conventional coating pan using a solution of ethylcellulose and HPMC E50 in a mixture of dichloromethane and methanol. The tablets that release oxybutynin at predetermined time of about 4 hours could be obtained when the tablets were coated to a weight gain of 13-14% by weight of the core and the tablets that release oxybutynin at a predetermined time of about 8 hours could be obtained when the tablets were coated to a weight gain of 20% by weight of the core.

One immediate release tablet, one tablet releasing oxybutynin at a predetermined time of about 4 hours and one tablet releasing oxybutynin at a predetermined time of about 8 hours were encapsulated in a hard gelatin capsule to provide an antispasmodic agent spaced drug delivery system of oxybutynin.

### EXAMPLE 2

The antispasmodic agent spaced drug delivery system of the present invention was prepared as per the formula in Table 4 below.

TABLE 4

| | Quantity/percent per tablet | | | | | |
|---|---|---|---|---|---|---|
| | | | Tablet cores for 4 | | Tablet cores for 8 hour | |
| | Immediate release | | hour timed pulse | | timed pulse release | |
| | uncoated tablet | | release composition | | composition | |
| Ingredients | mg | % w/w | mg | % w/w | mg | % w/w |
| Intragranular | | | | | | |
| Oxybutynin chloride | 2.5 | 2.77 | 4.0 | 4.44 | 3.5 | 3.88 |
| Microcrystalline cellulose | 50.0 | 55.56 | 50.0 | 55.56 | 50.0 | 55.56 |
| (Avicel PH 101) | | | | | | |
| Lactose monohydrate | 19.0 | 21.11 | 17.5 | 19.44 | 18.0 | 20.0 |
| Croscarmellose Sodium | 9.0 | 10.0 | 9.0 | 10.0 | 9.0 | 10.0 |
| (Ac-Di-Sol) | | | | | | |
| Maize starch (as 10% | 5.0 | 5.56 | 5.0 | 5.56 | 5.0 | 5.56 |
| starch paste) | | | | | | |
| Extragranular | | | | | | |
| Microcystalline cellulose | 2.0 | 2.22 | 2.0 | 2.22 | 2.0 | 2.22 |
| (Avicel PH 102) | | | | | | |
| Colloidal Silicon dioxide | 2.0 | 2.22 | 2.0 | 2.22 | 2.0 | 2.22 |
| (Aerosil 200) | | | | | | |
| Magnesium stearate | 0.5 | 0.56 | 0.5 | 0.56 | 0.5 | 0.56 |
| Total | 90 | 100 | 90 | 100 | 90 | 100 |

The cores for the immediate release tablets, tablets releasing oxybutynin at a predetermined time of 4 hours and the tablets releasing oxybutynin at a predetermined time of 8 hrs were prepared by the method of preparation as described in example 1.

The immediate release tablet cores were not coated while the tablet cores for releasing the drug at a predetermined time intervals were coated using the coating composition given in Table 5 below.

TABLE 5

| Ingredients | Quantity (% w/w) |
|---|---|
| Ethyl Cellulose | 3.75 |
| Hydroxypropyl methyl cellulose (HPMC E50) | 1.25 |
| Dichloromethane | q.s. |
| Methanol | q.s. |

The core tablets were coated in a conventional coating pan using a solution of ethyl cellulose and HPMC E50 in a mixture of dichloromethane and methanol. The tablets that release oxybutynin at predetermined time of about 4 hours could be obtained when the tablets were coated to a weight gain of 13-14% by weight of the core and the tablets that release oxybutynin at a predetermined time of about 8 hours could be obtained when the tablets were coated to a weight gain of 20% by weight of the core.

One immediate release tablet, one tablet releasing oxybutynin at a predetermined time of about 4 hours and one tablet releasing oxybutynin at a predetermined time of about 8 hours were encapsulated in a hard gelatin capsule. The capsule was subjected to a dissolution study using pH 4.5 acetate buffer at 37±0.5° C. in a USP Type II apparatus (rpm=50). The release profile for oxybutynin is recorded in Table 6 below.

TABLE 6

| Time (Hours) | % Oxybutynin released |
|---|---|
| 0.5 | 28 |
| 4 | 28 |
| 6 | 68 |
| 8 | 72 |
| 10 | 93 |
| 12 | 102 |

### EXAMPLE 3

The bioavailability of the antispasmodic agent spaced drug delivery system of the present invention (Oxybutynin chloride 10 mg TR capsules) and that of the extended release tablets of oxybutynin chloride available commercially (Ditropan XL, 10 mg tablets) were studied. A single-dose, open label, randomized, comparative and two-way crossover pharmacokinetic study with a fourteen days washout period, was undertaken for the same.

Oxybutynin chloride (SPARC, Mumbai,) 10 mg capsules was used as the test product and Ditropan XL (Alza Corporation, USA, Lot no. TF 332, Exp. Date: May 2002) 10 mg tablets was used as the reference product.

The pharmacokinetics assessment was based on the plasma levels of oxybutynin chloride measured by blood sampling. Blood samples were obtained before dosing and at the following time points after dosing of both the reference and the test products at 2, 4, 5, 6, 7, 8, 9, 10, 10.5, 11, 11.5, 12, 12.5, 13, 14, 24, 36 and 48 hours.

Six healthy male volunteers were enrolled for the study and all of them completed the two-way crossover study. The subjects fasted overnight before dosing and for 4 hours thereafter. Drinking water was prohibited 2 hours before dosing and 2 hours thereafter. Standard meals were provided at 4 hours and 8 hours after dosing and at appropriate times thereafter. Meal plans were identical for both the periods.

Subjects received a single oral dose of oxybutynin chloride 10 mg capsules (test product) and a single oral dose of Ditropan XL 10 mg tablets (reference product) with 240 ml of drinking water at ambient temperature after the overnight fast for the study.

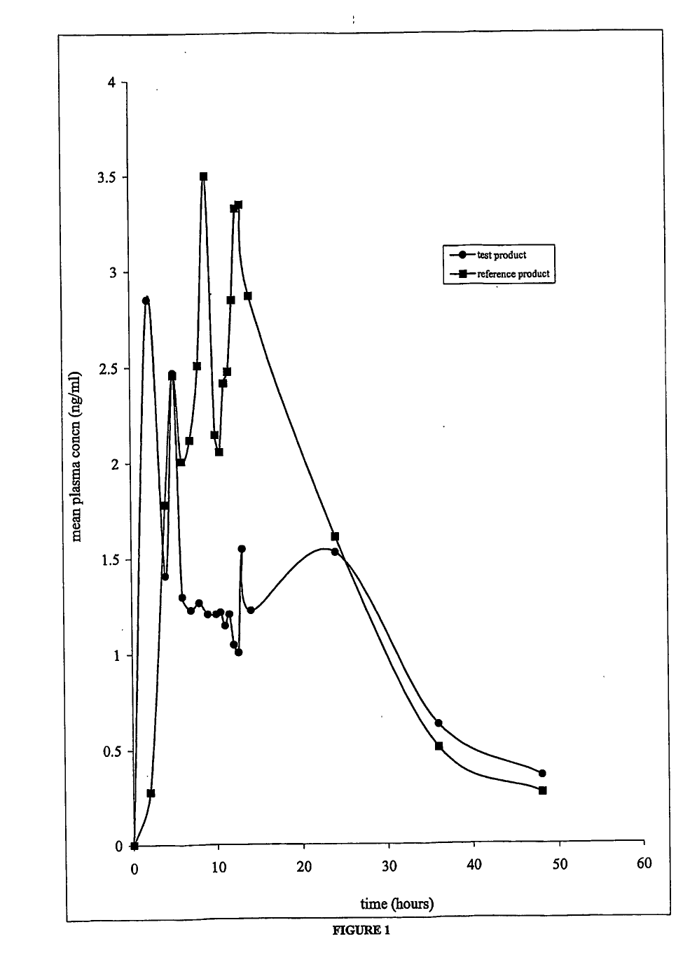

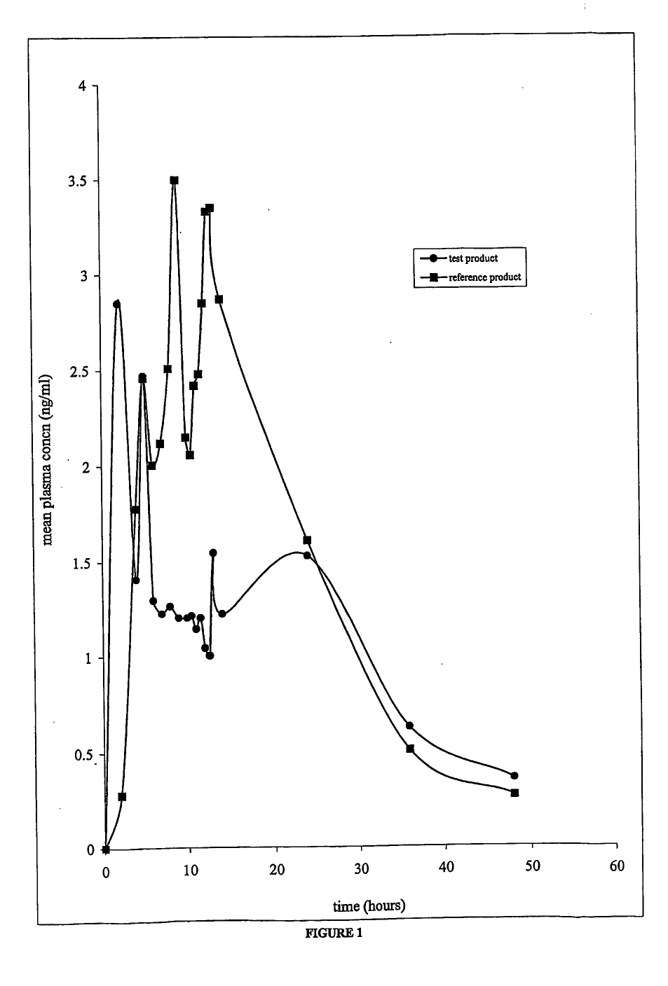

The plasma concentration of oxybutynin was determined for samples collected at different time points and averaged over the six volunteers. The data is given in Table 7 below. The plasma concentration versus time profile is illustrated in FIG. 1.

TABLE 7

| | Mean Plasma Concentrations (ng/ml) | |
|---|---|---|
| Time | Oxybutynin chloride 10 mg capsules | Ditropan XL 10 mg tablets |
| (hrs) | (Test product) | (Reference product) |
| 0.0 | 0.0 | 0.0 |
| 2.0 | 2.85 | 0.28 |
| 4.0 | 1.41 | 1.78 |
| 5.0 | 2.47 | 2.46 |
| 6.0 | 1.3 | 2.01 |
| 7.0 | 1.23 | 2.12 |
| 8.0 | 1.27 | 2.51 |
| 9.0 | 1.21 | 3.5 |
| 10.0 | 1.21 | 2.15 |
| 10.5 | 1.22 | 2.06 |
| 11.0 | 1.15 | 2.42 |
| 11.5 | 1.21 | 2.48 |
| 12.0 | 1.05 | 2.85 |
| 12.5 | 1.01 | 3.33 |
| 13.0 | 1.55 | 3.35 |
| 14.0 | 1.23 | 2.87 |
| 24.0 | 1.53 | 1.61 |
| 36.0 | 0.63 | 0.51 |
| 48.0 | 0.36 | 0.27 |

While the invention has been described with reference to specific embodiments, this was done for purposes of illustration only and should not be considered to limit the scope of the invention.

1. An antispasmodic agent spaced drug delivery system comprising;
(a) an immediate release composition comprising oxybutynin or its pharmaceutically acceptable salts and pharmaceutically acceptable excipients;
(b) a timed pulse release composition releasing oxybutynin or its pharmaceutically acceptable salts in a pulse at about a predetermined time; and
(c) optionally further comprising timed pulse release composition(s) as defined in (b) above, and releasing oxybutynin or its pharmaceutically acceptable salts in a pulse at about a predetermined time which is different from that of the timed pulse release composition defined in (b).

2. An antispasmodic agent spaced drug delivery system comprising;
(a) an immediate release composition comprising oxybutynin or its pharmaceutically acceptable salts and pharmaceutically acceptable excipients;
(b) a timed pulse release composition comprising a core comprising oxybutynin or its pharmaceutically acceptable salts, a swelling agent that swells to at least twice its volume upon imbibing water from the environment, and optionally, water-soluble compound(s) for inducing osmosis; and a coat surrounding the core wherein the coat comprises coating agents selected and used in amounts such that the coat ruptures or bursts to release in a pulse, the oxybutynin or its pharmaceutically acceptable salts at about a predetermined time; and
(c) optionally further comprising timed pulse release composition(s) as defined in (b) above, but which release(s) oxybutynin or its pharmaceutically acceptable salts in a pulse at about a predetermined time which is different from that of the timed pulse release composition defined in (b).

3. An antispasmodic agent spaced drug delivery system as claimed in claim 2 wherein the swelling agent is selected from the group consisting of crosslinked sodium carboxymethyl cellulose, crosslinked polyvinylpyrrolidone and sodium starch glycolate.

4. An antispasmodic agent spaced drug delivery system as claimed in claim 2 wherein the coating agent comprises a mixture of at least two coating agents.

5. An antispasmodic agent spaced drug delivery system as claimed in claim 4 wherein the coating agent comprises a mixture of a water-insoluble polymer and water-soluble compound selected from a water-soluble plasticizer and water-soluble polymer.

6. An antispasmodic agent spaced drug delivery system as claimed in claim 5 wherein the coating agent comprises a mixture of ethyl cellulose and hydroxypropyl methylcellulose.

7. An antispasmodic agent spaced drug delivery system as claimed in claim 2 wherein the swelling agent is croscarmellose sodium.

8. An antispasmodic agent spaced drug delivery system as claimed in claim 2 wherein the timed pulse release composition further comprises a wicking agent.

9. An antispasmodic agent spaced drug delivery system as claimed in claim 8 wherein the wicking agent is selected from the group consisting of microcrystalline cellulose and colloidal silicon dioxide or mixtures thereof.

10. An antispasmodic agent spaced drug delivery system as claimed in claim 2 wherein the core comprises the following:

| | | Range of amount used |
|---|---|---|
| | | as percent by weight of |
| Components | Name | core |
| Antispasmodic | Oxybutynin chloride | 2-5% |
| agent | | |
| Swelling agent | Croscarmellose sodium | 5-15% |
| Wicking agent | Microcrystalline cellulose | 40-60% |
| | Colloidal silicon dioxide | 1-30% |
| Water soluble | Lactose | 15-25% |
| compound | | |

and optionally other pharmaceutically acceptable excipients

11. An antispasmodic agent spaced drug delivery system as claimed in claim 10 wherein the coat surrounding the core comprises coating agent comprising a mixture of ethyl cellulose and hydroxypropyl methylcellulose in ethyl cellulose: hydroxypropyl methylcellulose ratios by weight from about 4:1 to 2:1.

12. An antispasmodic agent spaced drug delivery system as claimed in claim 11 wherein the coating agents are used in an amount from about 12 to 15% by weight of the core such that the predetermined time of release is about 4 hours.

13. An antispasmodic agent spaced drug delivery system as claimed in claim 11 wherein the coating agents are used in an amount from about 18 to 22% by weight of the core such that the predetermined time of release is about 8 hours.

14. An antispasmodic agent spaced drug delivery system as claimed in claim 2 comprising;
(a) an immediate release composition comprising oxybutynin or its pharmaceutically acceptable salts and pharmaceutically acceptable excipients;
(b) a first timed pulse release composition releasing oxybutynin or its pharmaceutically acceptable salts in a pulse at about 4 hours; and
(c) a second timed pulse release composition releasing oxybutynin or its pharmaceutically acceptable salts in a pulse at about 8 hours.
